(19) United States
(12) Patent Application Publication
(10) Pub. No.: US 2002/0127728 A1
(43) Pub. Date: Sep. 12, 2002

(54) Exhaust monitoring method

(76) Inventor: Kenji Izaki, Naka-gun (JP)

Correspondence Address: WENDEROTH, LIND & PONACK, L.L.P., 2033 K STREET N. W., SUITE 800, WASHINGTON, DC, 20006-1021, US

(21) Appl. No.: 09/984,234

(22) Filed: Oct. 29, 2001

(30) Foreign Application Priority Data
Jan. 5, 2001 (JP) 2001-544

(51) Int. Cl. G01N 33/00

(52) U.S. Cl. 436/57; 436/177

(57) ABSTRACT

An exhaust monitoring method for evaluating and monitoring a radioactive material concentration in exhaust by sampling the exhaust from radioactive materials handling facilities, collecting dust contained in the exhaust with an exhaust filter medium for monitoring, and performing the radioactivity measurement of the exhaust filter medium. The method is characterized in that a HEPA filter is disposed in an exhaust duct, the exhaust is sampled from the exhaust duct immediately after being filtered by the HEPA filter, and an exhaust sampling path is provided such that the sampled exhaust reaches the exhaust filter medium for monitoring within 20 seconds after passing through the HEPA filter, whereby a background for the radioactivity measurement is reduced. By performing the nuclide analysis of the exhaust filter medium, the abnormal/normal state of an exhaust path from the HEPA filter to the exhaust filter medium can be judged based on the presence/absence of the progenies of radon.

### BACKGROUND OF THE INVENTION

The present invention relates to a method for evaluating and monitoring a radioactive material concentration in exhaust from radioactive materials handling facilities, and more particularly to an exhaust monitoring method which can reduce a background (count derived from natural radio-nuclides) of an exhaust monitor by arranging a location where the exhaust monitor samples the exhaust immediately after a HEPA (High Efficiency Particulate Air) filter thus enabling the more accurate evaluation of the radioactive material concentration in the exhaust (the radioactive material concentration of only materials handled in the facilities).

In facilities which handle the radioactive material such as nuclear fuel materials handling facilities, radioactive isotope handling facilities or spent fuel reprocessing facilities, to administer gaseous waste, the radioactive material concentration in the exhaust is monitored with the use of an exhaust monitor.

As shown in FIG. 6, for example, in nuclear fuel material handling facilities 10, a gaseous waste discharged from a process chamber 12 or a glove box 14 or the like passes through an exhaust duct 18 having a HEPA filter 16 therein. Dust (minute particles) is removed by the HEPA filter 16 and is discharged into atmosphere through an exhaust flue 20. The exhaust gas is sampled from the exhaust flue 20 and the radioactive material concentration in the exhaust is measured by means of an exhaust monitor 30.

In the prior art, a sampling pipe 32 for exhaust monitoring is connected to a portion of the exhaust flue 20 in the vicinity of a distal end thereof. A portion of the exhaust is taken into the exhaust monitor 30 through the sampling pipe 32 and is returned to the exhaust flue 20 through a return pipe 36 after passing through an exhaust filter medium (usually, a filter paper 34). Thus, dust contained in the exhaust is collected by the exhaust filter paper 34 for monitoring and the radioactive measurement of the exhaust filter paper 34 is performed whereby the radioactive material concentration in the exhaust is evaluated and monitored.

In the exhaust from the various radioactive materials handling facilities, natural radioactive materials (radon: Rn-222, thoron: Rn-220, or progenies thereof) are contained. Accordingly, due to the influence derived from these natural radioactive materials, in spite of the fact that the radioactive materials which are handled in the inside of the facilities are not emitted to the outside of the facilities, an indication value of the exhaust monitor indicates several cpm. However, depending on the facilities, there is also a case where the background (here, the count derived from natural radio-nuclides) of the exhaust monitor reaches several tens cpm. Further, the indication value of the exhaust monitor fluctuates due to influences of a ventilation equipment and the like. Due to such influences, there arises a possibility that when the exhaust monitor exhibits the abnormal state actually, not only it is impossible to rapidly judge a cause thereof (an emission of the radioactive materials into the atmosphere, a defect of a detector or an elevation of the count due to noise or the like), but also even the detection of the abnormal state becomes impossible.

When an accident or a trouble occurs in the radioactive materials handling facilities by chance, to promptly cope with the accident or the trouble, it is important to accurately detect the abnormal state and to accurately and promptly specify the cause of the abnormal state. However, with respect to the above-mentioned conventional method, particularly in the facilities in which the background of the exhaust monitor is high, the accurate evaluation of the radioactive material concentration in the exhaust (the evaluation of the radioactive material concentration of only the materials handled in the inside of the facilities) is difficult.

### SUMMARY OF THE INVENTION

Accordingly, it is an object of the present invention to provide an exhaust monitoring method which can reduce the background of the exhaust monitor so that the evaluation of the radioactive material concentration in the exhaust (the evaluation of the radioactive material concentration of only the materials handled in the inside of the facilities) can be performed more accurately even in the facilities in which the background of the exhaust monitor is high.

It is another object of the present invention to provide a method which can easily judge an abnormal or normal state of an exhaust path from the HEPA filter of the exhaust duct to the exhaust filter medium of the exhaust monitor.

With respect to the background of the exhaust monitor, since it has been considered that the background is inevitably generated, any specific technique to reduce the background has not been reviewed heretofore. However, to fundamentally solve the above-mentioned problem of the prior art, it is considered most effective to reduce the background of the exhaust monitor. Accordingly, inventors of the present invention have analyzed the behavior of the natural radio-nuclides in the inside of the nuclear fuel materials handling facilities, have found based on the analysis that the background of the exhaust monitor can be reduced by changing the exhaust sampling position of the exhaust monitor, and have completed the present invention.

The present invention is directed to an exhaust monitoring method for evaluating and monitoring a radioactive material concentration in exhaust by sampling the exhaust from radioactive materials handling facilities, collecting dust contained in the exhaust with an exhaust filter medium for monitoring, and performing the radioactivity measurement of the exhaust filter medium. The present invention is characterized in that a HEPA filter is disposed in an exhaust duct, the exhaust is sampled from the exhaust duct immediately after being filtered by the HEPA filter, and an exhaust sampling path is provided such that the sampled exhaust reaches the exhaust filter medium for monitoring within 20 seconds (approximately ⅓ of the half-life of thoron) after passing through the HEPA filter, whereby a background for the radioactivity measurement is reduced.

Further, the present invention is also directed to an exhaust monitoring method according to the above-described method, wherein by performing a nuclide analysis of the exhaust filter medium after collecting the dust, an abnormal or normal state of an exhaust path from the HEPA filter to the exhaust filter medium is judged based on the presence or the absence of progenies of radon.

### BRIEF DESCRIPTION OF THE DRAWINGS

FIG. 1 is an operational explanatory view of a HEPA filter in an exhaust duct.

FIG. 2 is an operational explanatory view of an exhaust filter paper provided to a sampling pipe.

FIG. 3 is an explanatory view showing one embodiment of an exhaust monitoring method according to the present invention.

FIG. 4 is an explanatory view showing another embodiment of the exhaust monitoring method according to the present invention.

FIG. 5 is a graph showing the probability that thoron exists as progenies thereof after passing through a HEPA filter.

FIG. 6 is an explanatory view showing an example of a conventional exhaust monitoring method.

### PREFERRED EMBODIMENTS OF THE INVENTION

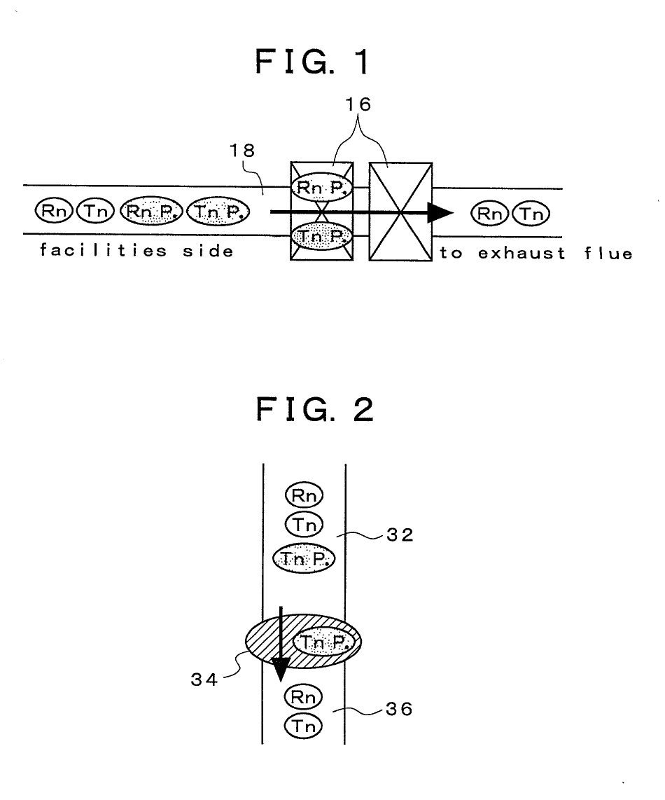

As shown in FIG. 1, in air filled in the inside of radioactive materials handling facilities, radon “Rn” (Rn-222) and thoron “Tn” (Rn-220) in a gaseous form and radon progenies “Rn P.” and thoron progenies “Tn P.” in a dust form are contained in mixture as natural radio-nuclides. However, in the radioactive materials handling facilities, when the air in the inside of the facilities is discharged to the outside of the facilities through an exhaust duct 18, particles are usually removed by making the exhaust pass through a HEPA filter 16. Therefore, in the exhaust immediately after being filterer by the HEPA filter 16, the progenies in a dust form are removed from natural radio-nuclides and hence, the progenies are not substantially present in the exhaust and only radon and thoron in a gaseous form are present in the exhaust.

Radon and thoron in a gaseous form contained in the air which has passed through the HEPA filter 16 are disintegrated or decayed as time lapses (in accordance with the half-life) and become progenies thereof. However, in a short period of time in which the exhaust passes through the HEPA filter 16 and then is emitted to the outside of the facilities, although there exists a probability that thoron which has the relatively short half-life (half-life : 55.6 seconds) is disintegrated and becomes progenies thereof, radon which has the relatively long half-life (half life: 3.8 days) is hardly disintegrated and it is emitted in a form of radon. To summarize the properties of these natural radio-nuclides, they can be listed as shown in Table 1. In Table 1, the half-life of progenies indicates the mean values of mixed nuclides.

TABLE 1

| | Half-life | State |
|---|---|---|
| radon(Rn-222) | 3.8 days | gaseous form |
| thoron(Rn-220) | 55.6 seconds | |
| progenies of radon | about 35 minutes | dust form |
| progenies of thoron | about 11 hours | |

An exhaust monitor collects dust contained in the air in the inside of an exhaust flue (or in the inside of the exhaust duct) using an exhaust filter medium, usually an exhaust filter paper, and the radioactive material concentration in the exhaust can be evaluated by measuring the radioactivity of the exhaust filter paper. Accordingly, as shown in FIG. 2, in the exhaust taken into the exhaust monitor through a sampling pipe 32, nuclides which are adhered to the exhaust filter paper 34 and accordingly are detected as the background of the exhaust monitor are only the progenies in a dust form among the natural radio-nuclides and only the progenies of thoron are present on the exhaust filter paper.

From the explanation described above, it is understood that to reduce the background of the exhaust monitor, it is necessary to decrease the progenies of thoron which are Adhered to the exhaust filter paper. That is, it is necessary to perform the sampling of the exhaust during a period in which thoron is not disintegrated to become progenies thereof. This can be realized by performing the sampling of the exhaust gas immediately after the HEPA filter.

Further, when the exhaust sampling is performed immediately after the HEPA filter, the progenies of radon are not collected by the exhaust filter paper in the normal state. Accordingly, by carrying out the nuclide analysis of the exhaust filter paper, it becomes possible to judge the abnormal or normal state of an exhaust path from the HEPA filter to the exhaust filter paper and hence, the leakage of an exhaust system can be detected. That is, when the progenies of radon are adhered to the exhaust filter paper, it is recognized that the air in the inside of the facilities (the air containing the progenies of radon) is mixed into the inside of the exhaust system. Here, “the inside of the exhaust system” means a pipe of the exhaust duct which is extended from a position where the exhaust passes through the HEPA filter to the exhaust filter paper. As phenomena which can be judged, a leakage of the exhaust duct (a hole being formed in the duct so that air outside the duct enters the inside of the duct), an insufficient mounting of the HEPA filter, a leakage of the sampling pipe of the exhaust monitor, an insufficient mounting of the exhaust filter paper and the like are considered.

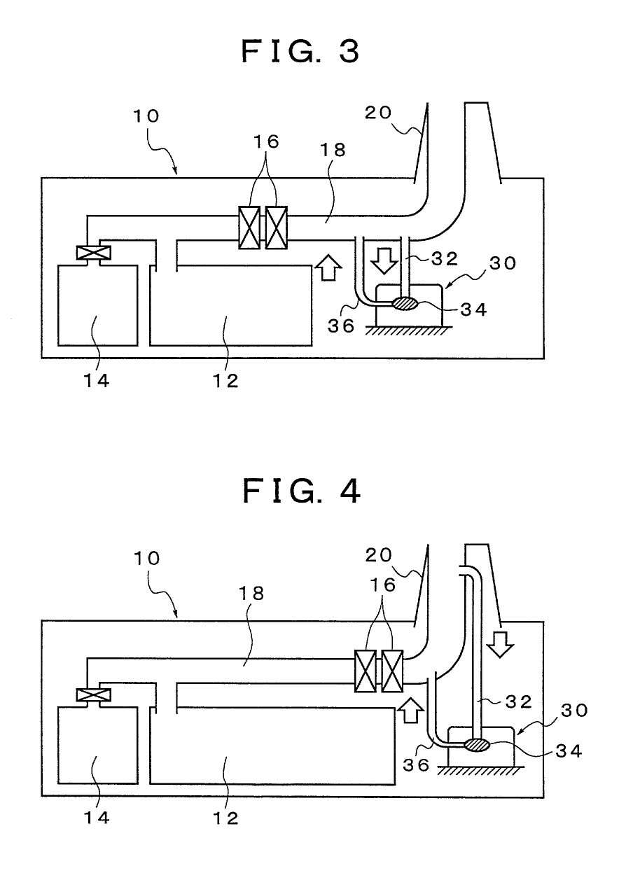

FIG. 3 and FIG. 4 are explanatory views showing embodiments of an exhaust monitoring method according to the present invention. To facilitate the understanding, portions similar to those shown in FIG. 6 are indicated by same symbols.

In nuclear fuel materials handling facilities 10, gaseous waste discharged from a process chamber 12 or a glove box 14 passes through an exhaust duct 18 having a HEPA filter 16 and is discharged into the atmosphere through an exhaust flue 20. In the course of this process, dust (fine particles) is removed by the HEPA filter 16. According to the present invention, an exhaust sampling path is set such that the exhaust sampling is performed by the exhaust monitor 30 immediately after the HEPA filter 16 which is disposed in the exhaust duct 18 and the sampled exhaust reaches an exhaust filter paper 34 for monitoring within a period of time which is equal to or less than 20 seconds (approximately ⅓ of the half-life of the thoron) after passing through the HEPA filter 16.

Although the embodiment shown in FIG. 3 sets the position of the HEPA filter 16 in the exhaust duct 18 in the same manner as the prior art shown in FIG. 6, a sampling pipe 32 of the exhaust monitor 30 is connected to the exhaust duct 18 immediately after the HEPA filter 16. Further, in the embodiment shown in FIG. 4, the HEPA filter 16 is located at a position as rearward as possible in the exhaust duct 18 (that is, a position close to the exhaust flue 20) and the sampling pipe 32 of the exhaust monitor 30 is connected to the exhaust flue 20 immediately after the HEPA filter 16. In these embodiments, the exhaust monitor 30 is constituted such that it includes the sampling pipe 32 which takes the exhaust into the exhaust monitor 30, the exhaust filter paper 34 and a return pipe 36 which returns the sampled exhaust gas, and performs the radioactive measurement of the exhaust filter paper 34.

The mounting of the exhaust monitor 30 immediately after the HEPA filter 16 means that the distance from the HEPA filter 16 to the exhaust filter paper 34 is made as short as possible. Specifically, in view of the half-life (55.6 seconds) of thoron (Rn-220), the exhaust sampling path is set such that the exhaust reaches the exhaust filter paper for monitoring within a period of time of equal to or less than 20 seconds (approximately ⅓ of the half-life of the thoron) after passing through the HEPA filter 16 in the same manner as mentioned hereinbefore. However, it is preferable to set the exhaust sampling path such that the exhaust gas sampling can be carried out within a further sufficiently short period of time (for example, within approximately 10 seconds) compared to the half-life of thoron.

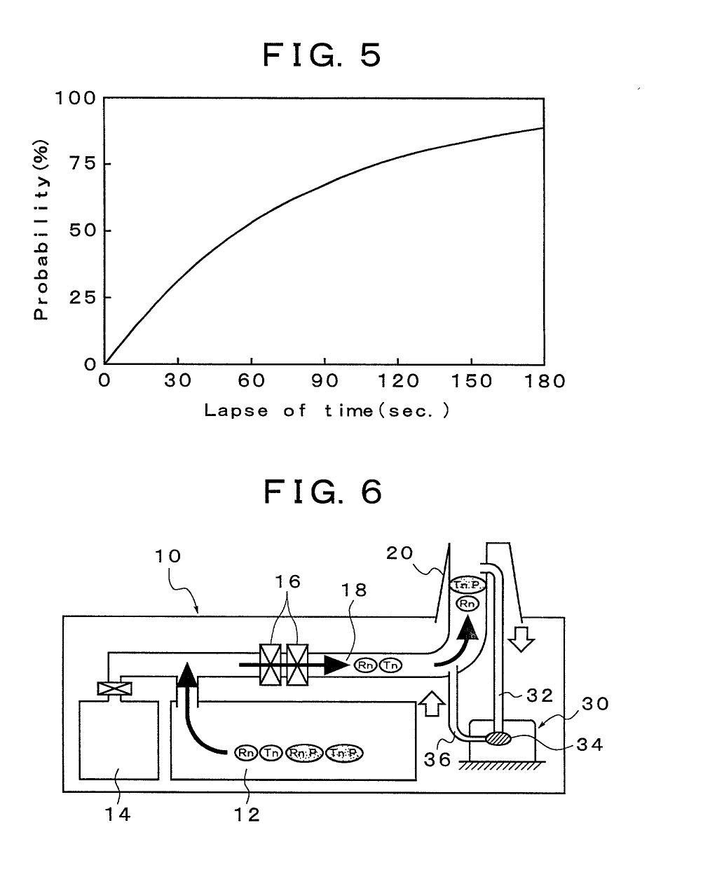

FIG. 5 shows the probability that thoron is disintegrated and exists as the progenies of thoron for a lapse of time from a point of time that the exhaust passes through the HEPA filter. At a point of time that 20 seconds (approximately ⅓ of the half-life) have lapsed, the probability is approximately 22%. The shorter the lapse of time, the existence probability of the progenies of thoron is decreased. That is, by performing the exhaust sampling within a sufficiently short period of time for the half-life of thoron, it becomes possible to largely reduce the background of the exhaust monitor. For example, by performing the sampling in approximately 5 seconds, which has conventionally been performed in approximately 75 seconds, the background can be reduced to 1/10 of the conventional background.

In the actual operation, the period of time that the exhaust reaches the exhaust filter paper from the HEPA filter is subjected to restrictions derived from the position of the HEPA filter and the structure of the exhaust duct at the time of installation designing of the facilities or the like and hence, it is important to take the method of the present invention into consideration at the time of designing. Further, in the facilities which have been already in service, besides the method which changes the exhaust sampling position to the position immediately after the HEPA filter as in the case of the above-mentioned embodiment, there may be considered a method which mounts an additional HEPA filter on the exhaust duct at a position immediately before the exhaust flue as an additional filtering stage. Further, there may be a case that a method which accelerates a flow speed of the exhaust gas is adopted.

In the method of the present invention, by not giving a sufficient period of time which is necessary for radon (Rn-222) and thoron (Rn-220) to be disintegrated to become progenies thereof in the inside of the facilities, the background of the air in the inside of the facilities can be reduced. To effectively accomplish the present invention, the number of locations where the air dwells may be decreased and, at the same time, the ventilation may be sufficiently performed.

As having been described hereinabove, since the present invention provides the method which reduces the background of the exhaust monitor by performing the exhaust sampling immediately after the HEPA filter, it becomes possible to promptly find out the presence or absence of emission of radioactive materials (radioactive materials handled in the inside of the facilities) to the outside of the facilities. Thus, the evaluation of the concentration of the radioactive materials in the exhaust (the concentration of the radioactive materials handled in the inside of the facilities) can be accurately and promptly performed. Further, along with the reduction of the background, it becomes possible to dissolve the misunderstanding that the radioactive materials are leaked to the outside of the facilities in spite of the fact that the radioactive materials are not leaked to the outside of the facilities actually.

Further, by performing the nuclide analysis of the exhaust filter medium through which the exhaust sampled immediately after the HEPA filter is made to pass according to the present invention, it becomes possible to judge the abnormal or normal state of the exhaust path from the HEPA filter to the exhaust filter medium based on the presence or absence of progenies of radon so that the leakage of the exhaust system can be detected.

What is claimed is:

1. An exhaust monitoring method for evaluating and monitoring a radioactive material concentration in exhaust by sampling the exhaust from radioactive materials handling facilities, collecting dust contained in the exhaust with an exhaust filter medium for monitoring, and performing the radioactivity measurement of the exhaust filter medium,
characterized in that a HEPA filter is disposed in an exhaust duct, the exhaust is sampled from the exhaust duct immediately after being filtered by the HEPA filter, and an exhaust sampling path is provided such that the sampled exhaust reaches the exhaust filter medium for monitoring within 20 seconds after passing through the HEPA filter, whereby a background for the radioactivity measurement is reduced.

2. The exhaust monitoring method according to claim 1, wherein by performing a nuclide analysis of the exhaust filter medium after collecting the dust, an abnormal or normal state of an exhaust path from the HEPA filter to the exhaust filter medium is judged based on the presence or the absence of progenies of radon.
